(19) United States
(12) Patent Application Publication
(10) Pub. No.: US 2026/0053654 A1
(43) Pub. Date: Feb. 26, 2026

(54) TRIPLE FLEXION DEVICE WITH QUICK DISCONNECT

(71) Applicant: Philip Muccio, Ann Arbor, MI (US)

(72) Inventor: Philip Muccio, Ann Arbor, MI (US)

(21) Appl. No.: 19/303,148

(22) Filed: Aug. 18, 2025

Related U.S. Application Data
(60) Provisional application No. 63/684,920, filed on Aug. 20, 2024.

(51) Int. Cl. A61F 5/01; A44B 11/25

(52) U.S. Cl. CPC A61F 5/0113; A44B 11/25

(57) ABSTRACT

A system includes a knee cuff including a quick release attachment mechanism, a shoe wrap extension comprising a shoe wrap, and a foot wrap extension comprising a foot wrap. The shoe wrap extension is configured to be removably attachable to the quick release attachment mechanism of the knee cuff, and the foot wrap extension is configured to be removably attachable to the quick release attachment mechanism of the knee cuff.

### CROSS REFERENCE TO RELATED APPLICATIONS

This patent application claims the benefit of and priority to U.S. provisional patent application 63/684,920 filed on Aug. 20, 2024, the contents of which are incorporated herein in their entirety.

### TECHNICAL FIELD

This disclosure relates to wearable devices that facilitate the flexion of at least three joints during walking, and particularly to such a device that facilitates a quick change between use of a shoe wrap and use of a foot wrap.

### BACKGROUND

Paralysis of the lower extremity can result in the loss of hip and knee flexion (hip and knee drop) and ankle flexion (foot drop) during the swing phase of walking. The hip, knee and ankle flex to pick the leg up to clear the ground to prevent dragging of the foot or tripping. The muscles responsible for this activity can become paralyzed through injury, disease or non-use (disuse muscle atrophy) that results in the inability to pick the leg up while walking.

Triple flexion devices to address the needs of a person that has paralysis are known. For example, U.S. Pat. No. 11,857,395B2, issued on Jan. 2, 2024, to the Applicant of the present application, discloses such a device. The contents of U.S. Pat. No. 11,857,395B2 are incorporated herein in their entirety.

The device includes a shoe wrap, designed to be wrapped around the outside of a shoe of a user. To support use of the triple flexion device without shoes, a foot wrap, designed to support the sole of a foot of the user, can also be provided.

### SUMMARY

Users of a triple flexion device typically want to use the device both with and without shoes. Switching between a shoe wrap and a foot wrap can be time consuming and require critical adjustment. For example, to switch from the shoe wrap to the foot wrap, first, the shoe wrap must be disconnected from the shoe. Then, the connecting straps, connecting the shoe wrap to a knee cuff of the triple flexion device must be disconnected and the connecting straps for the foot wrap connected. Then, critically, the length of the connecting straps for the foot wrap must be adjusted to a length to offer a proper level of support to the foot. The process must be reversed when switching between the foot wrap and the shoe wrap.

This paper discloses a quick disconnect system for triple flexion devices that supports quickly switching between shoe wraps and foot wraps without the need to adjust the length of the support straps for each conversion.

### BRIEF DESCRIPTION OF DRAWINGS

FIG. 1 is an exploded view of an example triple flexion system including interchangeable shoe wrap extension and foot wrap extension.

FIG. 2 shows a user attaching a waist belt of an example triple flexion system.

FIG. 3 shows a user attaching a waist belt of the example triple flexion system of FIG. 2.

FIG. 4 shows a user extending upper-leg straps from the example triple flexion system of FIG. 2.

FIG. 5 shows a front view of the example triple flexion system of FIG. 2 fully attached to the user.

FIG. 6 shows a side view of the example triple flexion system of FIG. 2 fully attached to the user.

FIG. 7 shows a side view of the example triple flexion system of FIG. 2 with the user in a walking position.

FIG. 8 shows a user attaching a foot wrap of an example triple flexion system.

FIG. 9 shows the user attaching the foot wrap of the example triple flexion system of FIG. 8.

FIG. 10 shows the user attaching the foot wrap of the example triple flexion system of FIG. 8.

FIG. 11 shows the user attaching lower-leg straps from the foot wrap to the knee cuff of the example triple flexion system of FIG. 8.

FIG. 12 shows upper-leg straps connecting the knee cuff to the waist belt attachment pad of the example triple flexion system of FIG. 8 with the user in a sitting position.

FIG. 13 shows a front view of the example triple flexion system of FIG. 8, with the user standing.

FIG. 14 shows an example quick release buckle for use with an example triple flexion system.

### DESCRIPTION

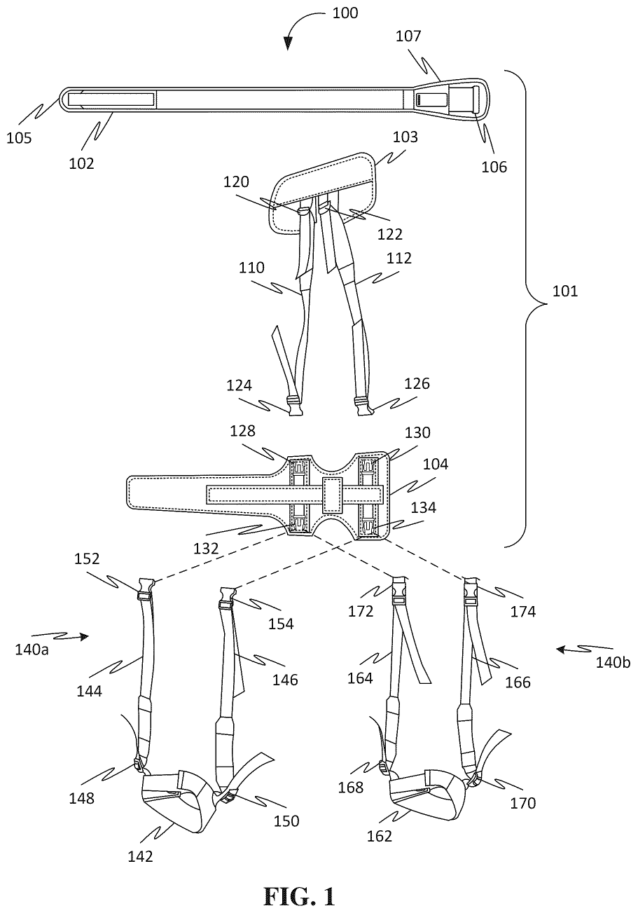

FIG. 1 shows an example triple flexion system 100. A triple flexion system 100 is made of conforming materials that fastens to the limb through a hook and loop fastener. Accordingly, the triple flexion system 100 is adjustable in size to fit a variety of people having a foot drop condition. In an example, the system 100 includes an upper-leg section 101, a shoe wrap extension 140a and a foot wrap extension 140b. As discussed below, the upper-leg section 101, the shoe wrap extension 140a and the foot wrap extension 140b are designed such that the shoe wrap extension 140a and the foot wrap extension 140b are removably attachable to the upper-leg section 101. This allows the shoe wrap extension 140a and the foot wrap extension 140b to be interchangeable with a minimum of effort.

The upper-leg section 101 includes a waist belt 102, a waist belt attachment pad 103, and a knee cuff 104. The waist belt 102 can be attached to the user by wrapping the waist belt 102 around the waist of the user and securing the belt by inserting a first end 105 of the waist belt 102 through a loop 106 on a second end 107 of the waist belt 102. After passing the first end 105 of the waist belt 102 through the loop 106, the first end 105 can be folded back on itself, and secured by, for example, a hook and loop attachment mechanism. The waist belt attachment pad 103 can then be secured to the waist belt 102 by use of, for example, a hook and loop attachment mechanism.

The upper-leg section 101 further includes two high-tension, elastic upper-leg straps 110, 112 extending from waist belt attachment pad 103 to the knee cuff 104. The upper-leg straps 110, 112 are adjustable by threading ends of the upper-leg straps 110, 112 respectively through loops 120, 122 to secure them to the waist belt attachment pad 103. The ends of the upper-leg straps 110, 112 are threaded respectively through the loops 120, 122 and then folded back on themselves, and may further be secured through a hook and loop fastening system. The figures show examples of two upper-leg straps being used. This is not intended to be limiting. In other examples a single upper-leg strap or more than two upper-leg straps may also be used.

A lower end of each of the upper-leg straps 110, 112 further includes a respective attachment mechanism 124, 126, for example a female portion of a quick release buckle, for attaching the lower end of the respective upper-leg strap 110, 112 to the knee cuff 104. The knee cuff can include two attachment mechanisms 128, 130, which can be, for example, male portions of the quick release buckles attached to the lower end of the respective upper-leg strap 110, 112, such that the upper-leg straps 110, 112 can buckle to the knee cuff 104, thereby forming an elastic connection between the waist belt attachment pad 103 and the knee cuff 104.

The knee cuff 104 is designed to be around the knee of the user. The knee cuff 104 can include, for example, hook and loop attachment mechanisms on ends of the knee cuff 104, to enable the knee cuff 104 to be wrapped around the knee of the user and securely held in place. The knee cuff 104 can further include two additional attachment mechanisms 132, 134, for attaching to either of the shoe wrap extension 140a or the foot wrap extension 140b of the triple flexion system 100.

The shoe wrap extension 140a includes a shoe wrap 142 and two high-tension, elastic lower-leg straps 144, 146.

The shoe wrap 142 is configured to wrap around and secure a shoe of the user. In an example, the shoe wrap 142 is a flexible band including a hook and loop attachment system to firmly wrap around a shoe of the user. The shoe wrap 142 includes two loops 148, 150 for connecting respectively to the lower-leg straps 144-146.

The lower-leg straps 144, 146 are designed to form an elastic connection between the knee cuff 104 and the shoe wrap 142. A lower end of each of the lower-leg straps 144, 146 can be threaded respectively through the loops 148, 150 on the shoe wrap 142. The ends of the lower straps 144, 146 are threaded respectively through the loops 148, 150 and then folded back on themselves.

Upper ends of the lower-leg straps 144, 146 include respective attachment mechanisms 152, 154, which, for example, can be female portions of respective quick release buckles, for attaching the upper ends of the respective lower-leg strap 144, 146 to the knee cuff 104. For example, the attachment mechanisms 152, 154 can be female portions of the attachment mechanisms 132, 134 on the knee cuff 104. In this manner, the shoe wrap extension 140a can be buckled to the knee cuff 104. As necessary, in order to exchange the shoe wrap extension 140a for the foot wrap extension 140b, the attachment mechanisms 152, 154 on the shoe wrap extension 140a can be unbuckled from the attachment mechanisms 132, 134 and the shoe wrap extension 140a disconnected from the knee cuff 104.

The figures show examples of two lower-leg straps being used in the shoe wrap extension 140a. This is not intended to be limiting. In other examples a single lower-leg strap or more than two lower-leg straps may also be used.

The foot wrap extension 140b includes a foot wrap 162 and two high-tension, elastic lower-leg straps 164, 166.

The foot wrap 162 is configured to wrap around and secure a foot of the user. In an example, the foot wrap 162 wrap is a flexible band including a hook and loop attachment system to firmly wrap around the foot of the user. The foot wrap 162 includes two loops 168, 170 for connecting respectively to the lower-leg straps 164-166.

The lower-leg straps 164, 166 are designed to form an elastic connection between the knee cuff 104 and the foot wrap 162. A lower end of each of the lower-leg straps 164, 166 can be threaded respectively through the loops 168, 170 on the wrap 162. The ends of the lower-leg straps 164, 166 are threaded respectively through the loops 168, 170 and then folded back on themselves.

Upper ends of the lower-leg straps include respective attachment mechanisms 172, 174, which, for example, can be female portions of respective quick release buckles, for attaching the upper ends of the respective lower-leg straps 164, 166 to the knee cuff 104. For example, the attachment mechanisms 172, 174 can be female portions of the attachment mechanisms 132, 134 on the knee cuff 104. In this manner, the foot wrap extension 140b can be buckled to the knee cuff 104. As necessary, in order to exchange the foot wrap extension 140b for the foot wrap extension 140a, the attachment mechanisms 172, 174 on the foot wrap extension 140b can be unbuckled from the attachment mechanisms 132, 134 and the foot wrap extension 140b thereby disconnected from the knee cuff 104.

The figures show examples of two lower-leg straps being used in the foot wrap extension 140b. This is not intended to be limiting. In other examples a single lower-leg strap or more than two lower-leg straps may also be used.

The example triple flexion system 100 has the advantage that a user can easily switch between using the shoe wrap extension 140a and the foot wrap extension 140b, or vice versa. For example, when the user comes home, the user can disconnect the shoe wrap extension 140a, remove the user's shoes, and attach the foot wrap extension 140b.

In addition to the simple process of unbuckling one extension and buckling in the other extension, the triple flexion system 100 offers the advantage that the shoe wrap extension 140a and the foot wrap extension 140b can be individually adjusted to provide the required support to the user, and do not need to be readjusted for each use. During an initial use of, for example, the shoe wrap extension 140a, a length of the lower-leg straps 144, 146 can be adjusted by use of the loops 148, 150 on the shoe wrap 142. An amount of the lower-leg straps 144, 146 that is fed through the loops can be adjusted until a desired tension is applied by the triple flexion system. The adjustment, once made, can remain the same.

Similarly, during an initial use of the foot wrap extension 140b, the length of the lower-leg straps 164, 166 can be adjusted by use of the loops 168, 170 on the foot wrap 162. An amount of the lower-leg straps 164, 166 that is fed through the loops 168, 170 can be adjusted until a desired tension is applied by the triple flexion system. This adjustment, once made, can also remain the same.

To make it easier to don the triple flexion system 100, a user can first put on the waist belt 102. As a next step, the user can put on either the shoe wrap extension 140a or foot wrap extension 140b. As a next step, the user can attach respectively the shoe wrap extension 140a or the foot wrap extension 140b to the knee cuff using the lower-leg straps 144, 146 in the case of the shoe wrap extension, or the lower-leg straps 164, 166 in the case of the foot wrap extension. As a next step, the user can connect the waist belt 102 to the knee cuff 104 using the waist belt attachment pad and the upper-leg straps 110, 112.

For using the toilet, the user can disconnect the waist belt 102 from the knee cuff 104 using the quick release attachment mechanism.

When wearing pants over the triple flexion system 100, the user can first put on the upper-leg section 101, and then the trousers pulled over the legs. Then, the pant legs of the trousers can be pulled up above the knee cuff 104, allowing either the shoe wrap extension 140a or the foot wrap extension 140b to be attached to the knee cuff 104.

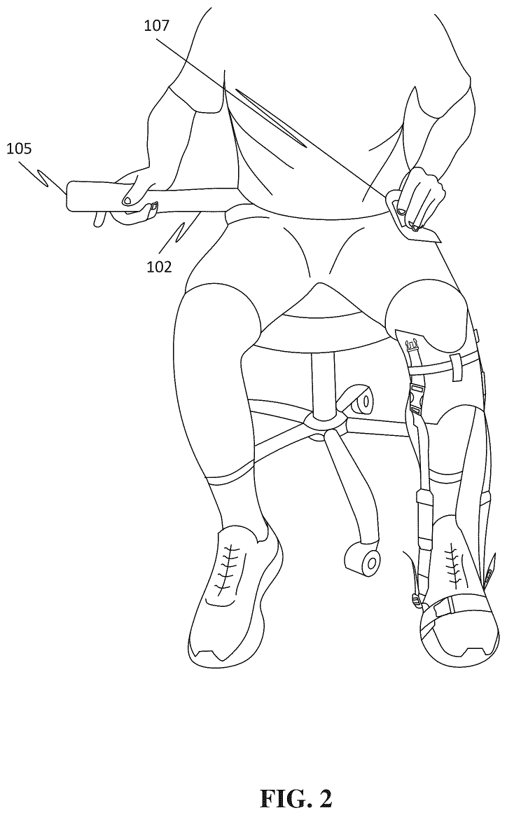

FIG. 2 shows a user wrapping an example waist belt 102 around the user's waist. The waist belt 102 has a first end 105 and a second end 107.

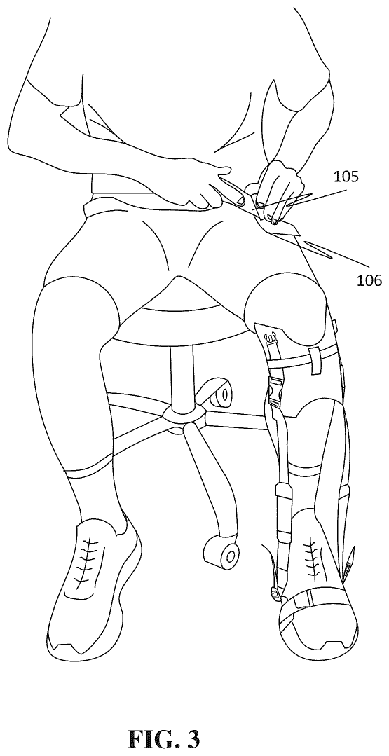

FIG. 3 shows the user securing the waist belt 102 by passing the first end 105 of the belt through a loop 106, folding the first end 105 back on itself and securing the end 105 using a hook and loop mechanism.

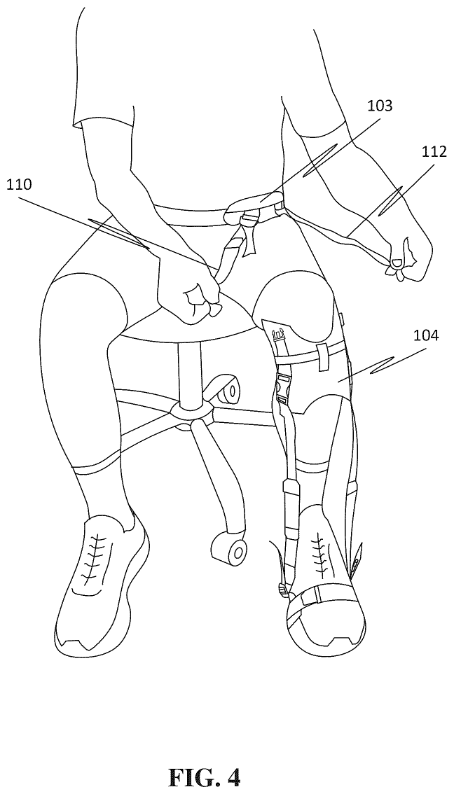

FIG. 4 shows the user extending upper-leg straps 110, 112 from the waist belt attachment pad 103 to the knee cuff 104.

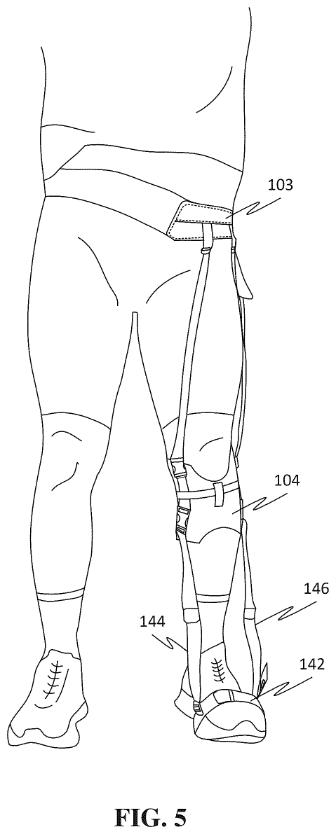

FIG. 5 shows the user in a standing position wearing the example triple flexion system 100, and showing the waist belt attachment pad 103, the knee cuff 104, the shoe wrap 142, and the lower-leg straps 144, 146.

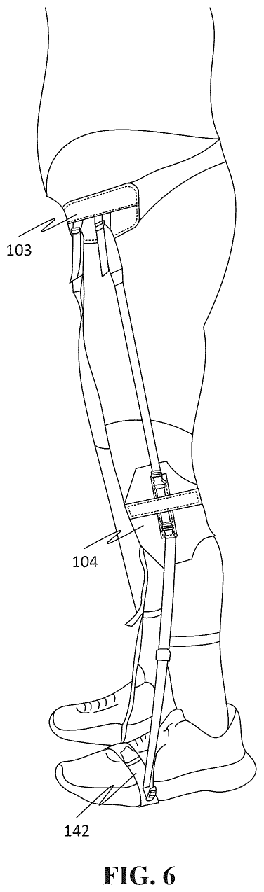

FIG. 6 shows a side view of the user in a standing position wearing the example triple flexion system 100, and showing the waist belt attachment pad 103, the knee cuff 104, and the shoe wrap 142.

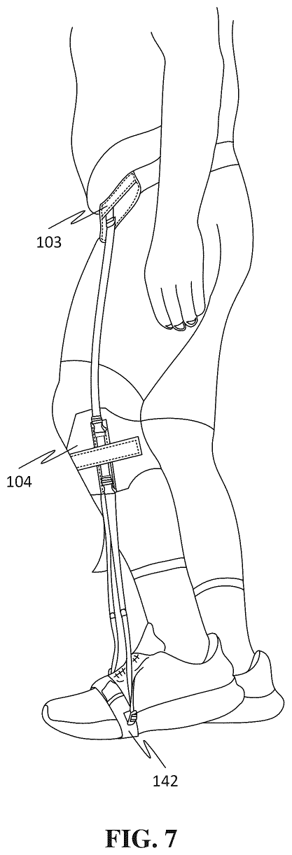

FIG. 7 shows a side view of the user in a walking position wearing the example triple flexion system 100, and showing the waist belt attachment pad 103, the knee cuff 104, and the shoe wrap 142.

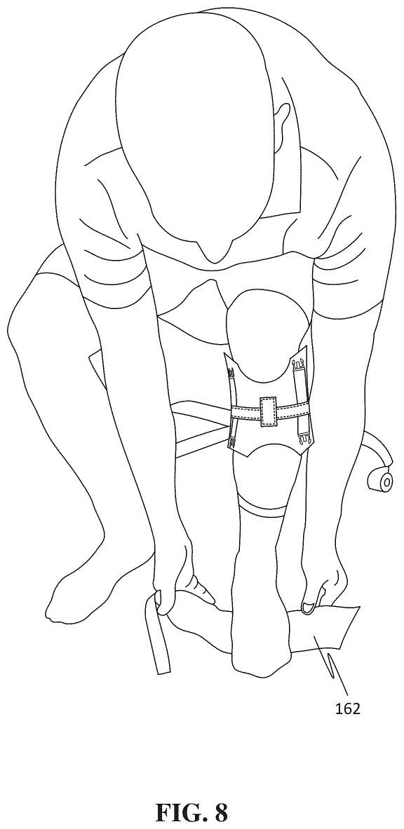

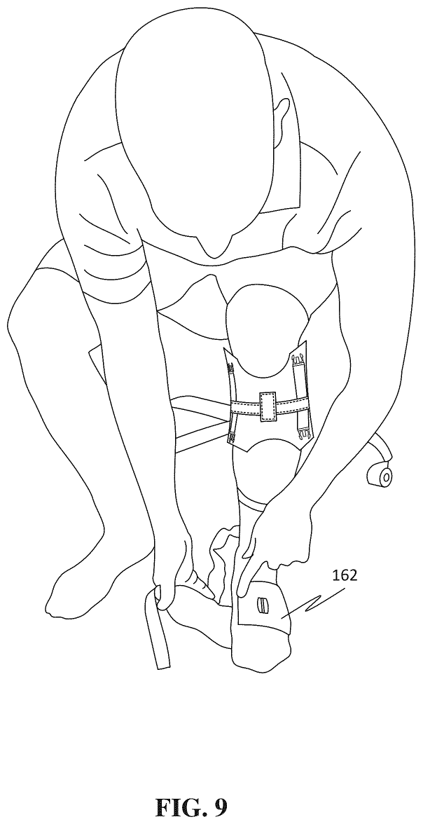

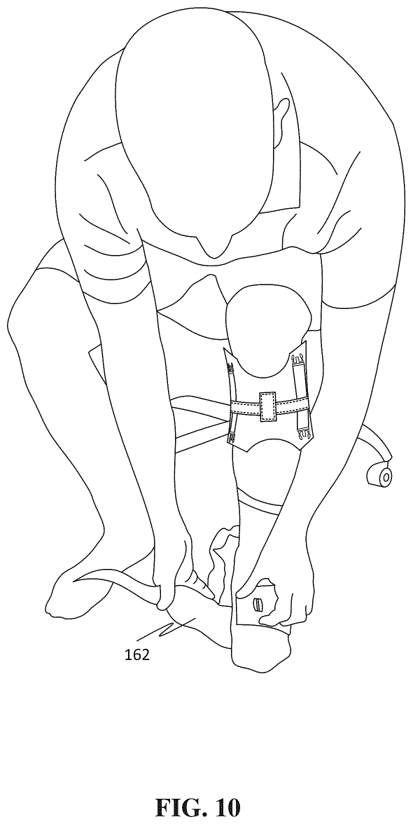

FIGS. 8, 9 and 10 show a user wrapping an example foot wrap 162 around the user's foot.

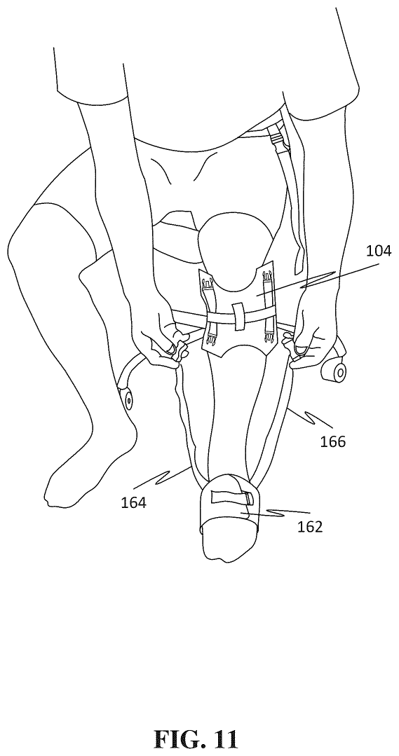

FIG. 11 shows the user extending lower-leg straps 164, 166 from the foot wrap 162 towards the knee cuff 104 of the triple flexion system 100.

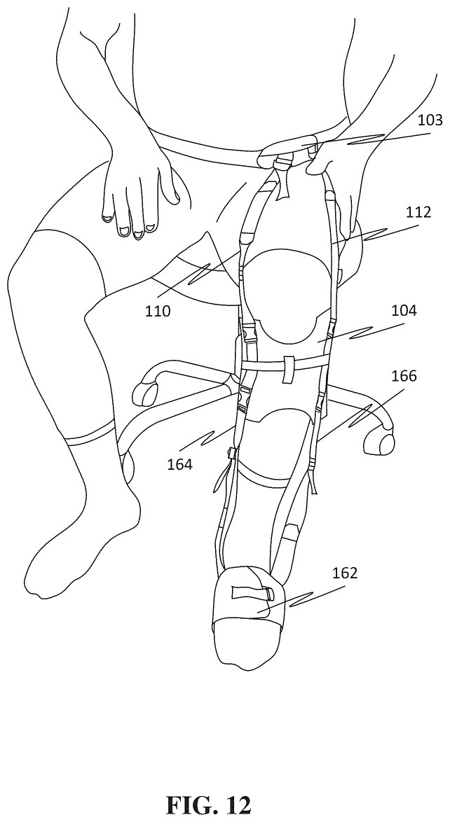

FIG. 12 shows the user in a sitting position wearing the triple flexion system 100 and showing the waist belt attachment pad 103, the knee cuff 104, the foot wrap 162, the upper-leg straps 110, 112 extending from the attachment pad 103 to the knee cuff 104, and the lower-leg straps 164, 166 extending from the knee cuff 104 to the foot wrap 162.

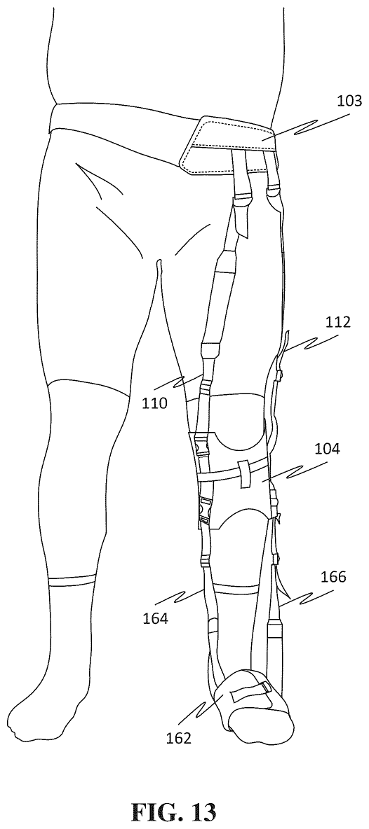

FIG. 13 shows the user in a standing position wearing the triple flexion system 100 and showing the waist belt attachment pad 103, the knee cuff 104, the foot wrap 162, the upper-leg straps 110, 112 extending from the attachment pad 103 to the knee cuff 104, and the lower-leg straps 164, 166 extending from the knee cuff 104 to the foot wrap 162.

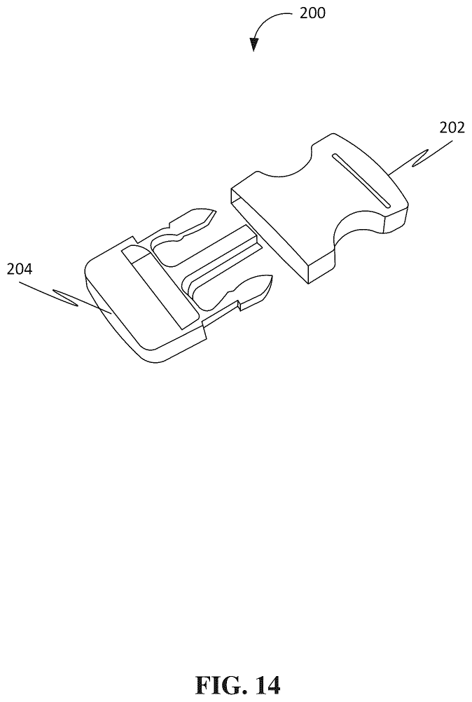

FIG. 14 shows an example quick release buckle 200. The quick release buckle includes a female portion 202 and a male portion 204. The quick release buckle 200 is an example of a quick release buckle that can be used for the attachment mechanisms 128, 130, 132, 134 on the knee cuff 104, the attachment mechanisms 152, 154 on the shoe wrap extension 140a and the attachment mechanisms 172, 174 on the foot wrap extension 140b.

What is claimed is:

1. A system comprising:
a knee cuff including a quick release attachment mechanism;
a shoe wrap extension comprising a shoe wrap; and
a foot wrap extension comprising a foot wrap; wherein:
the shoe wrap extension is configured to be removably attachable to the quick release attachment mechanism of the knee cuff; and
the foot wrap extension is configured to be removably attachable to the quick release attachment mechanism of the knee cuff.

2. The system of claim 1, wherein:
the foot wrap extension includes a foot wrap attachment mechanism configured to connect with the quick release attachment mechanism of the knee cuff; and
the shoe wrap extension includes shoe wrap attachment mechanism configured to connect to the quick release attachment mechanism of the knee cuff.

3. The system of claim 2, wherein either:
the quick release attachment mechanism of the knee cuff is a male portion of a quick release buckle, and both the foot wrap attachment mechanism and the shoe wrap attachment mechanism are matching female portions of the quick release buckle; or
the quick release attachment mechanism of the knee cuff is a female portion of a quick release buckle, and both the foot wrap attachment mechanism and the shoe wrap attachment mechanism are matching male portions of the quick release buckle.

4. The system of claim 2, wherein:
the shoe wrap extension further includes at least one shoe wrap lower-leg strap extending from the shoe wrap to the foot wrap attachment mechanism; and
the foot wrap extension further includes at least one foot wrap lower-leg strap extending from the foot wrap to the foot wrap attachment mechanism.

5. The system of claim 4, wherein:
at least a portion of the shoe wrap lower-leg strap is elastic; and
at least a portion of the foot wrap lower-leg strap is elastic.

6. The system of claim 1, further comprising:
a waist belt; and
a waist belt attachment pad; wherein, the waist belt is configured to be attached to the knee cuff via the waist belt attachment pad.

7. The system of claim 6, wherein the waist belt attachment pad is configured to attach to the waist belt via a hook and loop attachment mechanism.

8. The system of claim 6, wherein the knee cuff includes a second quick release attachment mechanism, and the waist belt attachment pad is configured to be removably attachable to the second quick release attachment mechanism of the knee cuff.

9. The system of claim 8, further comprising at least one upper-leg strap extending from the waist belt attachment mechanism, the upper-leg strap comprising an upper-leg strap attachment mechanism on a distal end, wherein the upper-leg attachment mechanism is configured to connect to the second quick release attachment mechanism of the knee cuff.

10. The system of claim 9, wherein either:
the second quick release attachment mechanism of the knee cuff is a male portion of a quick release buckle, and the upper-leg strap attachment mechanism is a matching female portion of the quick release buckle; or
the second quick release attachment mechanism of the knee cuff is a female portion of a quick release buckle, and the upper-leg strap attachment mechanism is a matching male portion of the quick release buckle.

11. A method comprising:
attaching a knee cuff to a knee of a user;
attaching a shoe wrap to a shoe of the user; and
attaching, using a quick release attachment mechanism, the shoe wrap to the knee cuff.

12. The method of claim 11, wherein the quick release attachment mechanism is a quick release buckle.

13. The method of claim 11, wherein at least one lower-leg strap extends from the shoe wrap, and the attachment mechanism connects a distal end of the at least one lower-leg strap to the knee cuff.

14. The method of claim 11, further comprising:
attaching a waist belt to the user;
attaching a waist belt attachment pad to the waist belt; and
attaching the knee cuff to the waist belt attachment pad using a second quick release attachment mechanism.

15. The method of claim 14, wherein the second quick release mechanism is a quick release buckle.

16. The method of claim 15, wherein at least one upper-leg strap extends from waist belt attachment pad, and the second quick release attachment mechanism connects a distal end of the at least one upper-leg strap to the knee cuff.

17. The method of claim 11, further comprising:
releasing the shoe wrap from the knee cuff using the quick release attachment mechanism; and
attaching a foot wrap to the knee cuff using the quick release attachment mechanism.

18. The method of claim 17, further comprising:
releasing the foot wrap from the knee cuff using the quick release attachment mechanism; and
attaching the shoe wrap to the knee cuff using the quick release attachment mechanism.
